SEQUENCE LISTING (174 sequences)

SEQ ID NO: 1 - DNA, 22 bp, synthetic construct
  source 1..22: mol_type = other DNA
ttgtactttgagaagggggagt

SEQ ID NO: 2 - DNA, 20 bp, synthetic construct
  source 1..20: mol_type = other DNA
ggtagcatgaggggatacgg

SEQ ID NO: 3 - DNA, 24 bp, synthetic construct
  source 1..24: mol_type = other DNA
aagaggagtttaaaaggctgaaca

SEQ ID NO: 4 - DNA, 19 bp, synthetic construct
  source 1..19: mol_type = other DNA
aaggtggcatcccataggc

SEQ ID NO: 5 - DNA, 20 bp, synthetic construct
  source 1..20: mol_type = other DNA
attggagacttcgggatggc

SEQ ID NO: 6 - DNA, 20 bp, synthetic construct
  source 1..20: mol_type = other DNA
cttaactggcagcatggcac

SEQ ID NO: 7 - DNA, 23 bp, synthetic construct
  source 1..23: mol_type = other DNA
ggccagtggagataacactctaa

SEQ ID NO: 8 - DNA, 23 bp, synthetic construct
  source 1..23: mol_type = other DNA
caccattccccattgtgattata

SEQ ID NO: 9 - DNA, 21 bp, synthetic construct
  source 1..21: mol_type = other DNA
agaagcaccttttcagcacca

SEQ ID NO: 10 - DNA, 23 bp, synthetic construct
  source 1..23: mol_type = other DNA
acactggatatttgagcctggat

SEQ ID NO: 11 - DNA, 20 bp, synthetic construct
  source 1..20: mol_type = other DNA
atgaagagctgacccagccg

SEQ ID NO: 12 - DNA, 20 bp, synthetic construct
  source 1..20: mol_type = other DNA
cctcataggactgggaccct

SEQ ID NO: 13 - DNA, 23 bp, synthetic construct
  source 1..23: mol_type = other DNA
gaacatagcacgtttctctctgg

SEQ ID NO: 14 - DNA, 22 bp, synthetic construct
  source 1..22: mol_type = other DNA
gtatcttgaaggggctcacttg

SEQ ID NO: 15 - DNA, 22 bp, synthetic construct
  source 1..22: mol_type = other DNA
ttcctcttcctcctactcacca

SEQ ID NO: 16 - DNA, 20 bp, synthetic construct
  source 1..20: mol_type = other DNA
ctggtgtcgttttgtcctga

SEQ ID NO: 17 - DNA, 20 bp, synthetic construct
  source 1..20: mol_type = other DNA
tttgtggccctcaagagtgt

SEQ ID NO: 18 - DNA, 20 bp, synthetic construct
  source 1..20: mol_type = other DNA
acgtccatcagccggacaac

SEQ ID NO: 19 - DNA, 20 bp, synthetic construct
  source 1..20: mol_type = other DNA
tcttgctccagtccagctac

SEQ ID NO: 20 - DNA, 21 bp, synthetic construct
  source 1..21: mol_type = other DNA
gggagtccaatcacgtccaag

SEQ ID NO: 21 - DNA, 19 bp, synthetic construct
  source 1..19: mol_type = other DNA
ggacatcagagggtcgctt

SEQ ID NO: 22 - DNA, 21 bp, synthetic construct
  source 1..21: mol_type = other DNA
gatgggagtccaggagaaaat

SEQ ID NO: 23 - DNA, 17 bp, synthetic construct
  source 1..17: mol_type = other DNA
gacgctgcgcacaacat

SEQ ID NO: 24 - DNA, 24 bp, synthetic construct
  source 1..24: mol_type = other DNA
ctggtcttcaacttccatttcatc

SEQ ID NO: 25 - DNA, 20 bp, synthetic construct
  source 1..20: mol_type = other DNA
gtacccaccgccatactacc

SEQ ID NO: 26 - DNA, 21 bp, synthetic construct
  source 1..21: mol_type = other DNA
gcattttcacatagacccctg

SEQ ID NO: 27 - DNA, 19 bp, synthetic construct
  source 1..19: mol_type = other DNA
gttcaaggatggcgatcag

SEQ ID NO: 28 - DNA, 21 bp, synthetic construct
  source 1..21: mol_type = other DNA
ggccagcatatcacacaatgt

SEQ ID NO: 29 - DNA, 23 bp, synthetic construct
  source 1..23: mol_type = other DNA
cccaggaaactcctaactttcaa

SEQ ID NO: 30 - DNA, 21 bp, synthetic construct
  source 1..21: mol_type = other DNA
tccctccacttcacagagatg

SEQ ID NO: 31 - DNA, 20 bp, synthetic construct
  source 1..20: mol_type = other DNA
gaacagcagtaccgagcctt

SEQ ID NO: 32 - DNA, 20 bp, synthetic construct
  source 1..20: mol_type = other DNA
acttctcgaacagaccgcag

SEQ ID NO: 33 - DNA, 17 bp, synthetic construct
  source 1..17: mol_type = other DNA
tccttaagcgcagccct

SEQ ID NO: 34 - DNA, 21 bp, synthetic construct
  source 1..21: mol_type = other DNA
aggacatgaaaccagacagga

SEQ ID NO: 35 - DNA, 23 bp, synthetic construct
  source 1..23: mol_type = other DNA
gggcttttatacggctcacagac

SEQ ID NO: 36 - DNA, 20 bp, synthetic construct
  source 1..20: mol_type = other DNA
aatggttctcctccagcctt

SEQ ID NO: 37 - DNA, 20 bp, synthetic construct
  source 1..20: mol_type = other DNA
gacgcaggggagtatacgtg

SEQ ID NO: 38 - DNA, 20 bp, synthetic construct
  source 1..20: mol_type = other DNA
tctcttccagggcttccaga

SEQ ID NO: 39 - DNA, 20 bp, synthetic construct
  source 1..20: mol_type = other DNA
cacgaccaagaagccagact

SEQ ID NO: 40 - DNA, 21 bp, synthetic construct
  source 1..21: mol_type = other DNA
tggactcagccgaaactgtta

SEQ ID NO: 41 - DNA, 19 bp, synthetic construct
  source 1..19: mol_type = other DNA
gaatgcctcccacgaggac

SEQ ID NO: 42 - DNA, 20 bp, synthetic construct
  source 1..20: mol_type = other DNA
gaggatggagcgtctgtcac

SEQ ID NO: 43 - DNA, 22 bp, synthetic construct
  source 1..22: mol_type = other DNA
ccgatgtatggtcatttggaat

SEQ ID NO: 44 - DNA, 19 bp, synthetic construct
  source 1..19: mol_type = other DNA
ctggattacctgggcacct

SEQ ID NO: 45 - DNA, 21 bp, synthetic construct
  source 1..21: mol_type = other DNA
ggttggtcaactggcagaaac

SEQ ID NO: 46 - DNA, 20 bp, synthetic construct
  source 1..20: mol_type = other DNA
agagctcctttcaggtctcg

SEQ ID NO: 47 - DNA, 19 bp, synthetic construct
  source 1..19: mol_type = other DNA
atcttcaccctgacacggg

SEQ ID NO: 48 - DNA, 21 bp, synthetic construct
  source 1..21: mol_type = other DNA
ttagagtcttcagccgtgctc

SEQ ID NO: 49 - DNA, 23 bp, synthetic construct
  source 1..23: mol_type = other DNA
tctgctactactacgacggtgat

SEQ ID NO: 50 - DNA, 20 bp, synthetic construct
  source 1..20: mol_type = other DNA
cagtggctttatggggccta

SEQ ID NO: 51 - DNA, 23 bp, synthetic construct
  source 1..23: mol_type = other DNA
tggcattgatgaccagagttaca

SEQ ID NO: 52 - DNA, 20 bp, synthetic construct
  source 1..20: mol_type = other DNA
tgcacgtgggttggtagaag

SEQ ID NO: 53 - DNA, 20 bp, synthetic construct
  source 1..20: mol_type = other DNA
ctggcgtcgtgattagtgat

SEQ ID NO: 54 - DNA, 21 bp, synthetic construct
  source 1..21: mol_type = other DNA
gagcaagacgttcagtcctgt

SEQ ID NO: 55 - DNA, 21 bp, synthetic construct
  source 1..21: mol_type = other DNA
tgctgcgaaagtcttcttgag

SEQ ID NO: 56 - DNA, 24 bp, synthetic construct
  source 1..24: mol_type = other DNA
tggctgttcagagaaatactcatc

SEQ ID NO: 57 - DNA, 20 bp, synthetic construct
  source 1..20: mol_type = other DNA
tgagccagaatgccttcatc

SEQ ID NO: 58 - DNA, 23 bp, synthetic construct
  source 1..23: mol_type = other DNA
agattcatctgtgtaatcaggcg

SEQ ID NO: 59 - DNA, 21 bp, synthetic construct
  source 1..21: mol_type = other DNA
gaatgcaagagaggtttccgc

SEQ ID NO: 60 - DNA, 20 bp, synthetic construct
  source 1..20: mol_type = other DNA
tgttccgagtggcagagctt

SEQ ID NO: 61 - DNA, 19 bp, synthetic construct
  source 1..19: mol_type = other DNA
acacttcctgccaagtcca

SEQ ID NO: 62 - DNA, 18 bp, synthetic construct
  source 1..18: mol_type = other DNA
atgcttgactcacgggga

SEQ ID NO: 63 - DNA, 20 bp, synthetic construct
  source 1..20: mol_type = other DNA
atttctggctggaacggacg

SEQ ID NO: 64 - DNA, 20 bp, synthetic construct
  source 1..20: mol_type = other DNA
gccagtcccttagacacacc

SEQ ID NO: 65 - DNA, 23 bp, synthetic construct
  source 1..23: mol_type = other DNA
cctcctactcctgaagacaacag

SEQ ID NO: 66 - DNA, 23 bp, synthetic construct
  source 1..23: mol_type = other DNA
tgaggatcattggtttggtagtc

SEQ ID NO: 67 - DNA, 20 bp, synthetic construct
  source 1..20: mol_type = other DNA
cacaggcatgccgtatctct

SEQ ID NO: 68 - DNA, 20 bp, synthetic construct
  source 1..20: mol_type = other DNA
ccagagcttggtgttctcgt

SEQ ID NO: 69 - DNA, 24 bp, synthetic construct
  source 1..24: mol_type = other DNA
tgctgcttctaaagcttgtggtat

SEQ ID NO: 70 - DNA, 21 bp, synthetic construct
  source 1..21: mol_type = other DNA
ggaagacatggttgggtggat

SEQ ID NO: 71 - DNA, 22 bp, synthetic construct
  source 1..22: mol_type = other DNA
tgacaatgtctcaccaagctct

SEQ ID NO: 72 - DNA, 20 bp, synthetic construct
  source 1..20: mol_type = other DNA
gaggacaatggacagagccc

SEQ ID NO: 73 - DNA, 21 bp, synthetic construct
  source 1..21: mol_type = other DNA
ccatctcaagctttcagagga

SEQ ID NO: 74 - DNA, 22 bp, synthetic construct
  source 1..22: mol_type = other DNA
tagcttctatcctttccaggga

SEQ ID NO: 75 - DNA, 20 bp, synthetic construct
  source 1..20: mol_type = other DNA
tgacacaccgtgagacaggt

SEQ ID NO: 76 - DNA, 21 bp, synthetic construct
  source 1..21: mol_type = other DNA
gcatgaccttcacctccttga

SEQ ID NO: 77 - DNA, 21 bp, synthetic construct
  source 1..21: mol_type = other DNA
gggagaatcttcaggtgactc

SEQ ID NO: 78 - DNA, 20 bp, synthetic construct
  source 1..20: mol_type = other DNA
ccaggaccaggacattcagt

SEQ ID NO: 79 - DNA, 19 bp, synthetic construct
  source 1..19: mol_type = other DNA
ccatcacctgcctcagaac

SEQ ID NO: 80 - DNA, 20 bp, synthetic construct
  source 1..20: mol_type = other DNA
aatacactgggagccagagc

SEQ ID NO: 81 - DNA, 19 bp, synthetic construct
  source 1..19: mol_type = other DNA
agctctgcggagaccaact

SEQ ID NO: 82 - DNA, 20 bp, synthetic construct
  source 1..20: mol_type = other DNA
gctgctgctcatcaagctct

SEQ ID NO: 83 - DNA, 23 bp, synthetic construct
  source 1..23: mol_type = other DNA
tatattgtgaacgagccacctcc

SEQ ID NO: 84 - DNA, 22 bp, synthetic construct
  source 1..22: mol_type = other DNA
tggtttgtgagcatcttcaggt

SEQ ID NO: 85 - DNA, 21 bp, synthetic construct
  source 1..21: mol_type = other DNA
cgaggacttcagcgaagtgta

SEQ ID NO: 86 - DNA, 20 bp, synthetic construct
  source 1..20: mol_type = other DNA
gccgagtggatgtacttcag

SEQ ID NO: 87 - DNA, 23 bp, synthetic construct
  source 1..23: mol_type = other DNA
ccaaacgaggagaggaattaaag

SEQ ID NO: 88 - DNA, 24 bp, synthetic construct
  source 1..24: mol_type = other DNA
gcctgtacagtttcatttggattt

SEQ ID NO: 89 - DNA, 24 bp, synthetic construct
  source 1..24: mol_type = other DNA
aagttttcttcaaaagagcagtgg

SEQ ID NO: 90 - DNA, 20 bp, synthetic construct
  source 1..20: mol_type = other DNA
tcgatgatgcaatcattcct

SEQ ID NO: 91 - DNA, 19 bp, synthetic construct
  source 1..19: mol_type = other DNA
tctggagctccgtgatctg

SEQ ID NO: 92 - DNA, 21 bp, synthetic construct
  source 1..21: mol_type = other DNA
agtgaaatggaaggcatctgg

SEQ ID NO: 93 - DNA, 20 bp, synthetic construct
  source 1..20: mol_type = other DNA
actttgctgggatcctgtcc

SEQ ID NO: 94 - DNA, 21 bp, synthetic construct
  source 1..21: mol_type = other DNA
caaacatgtagcctgtcacgg

SEQ ID NO: 95 - DNA, 21 bp, synthetic construct
  source 1..21: mol_type = other DNA
tcacaggttacatgtttggga

SEQ ID NO: 96 - DNA, 25 bp, synthetic construct
  source 1..25: mol_type = other DNA
ttcattacactgacctagagctacc

SEQ ID NO: 97 - DNA, 20 bp, synthetic construct
  source 1..20: mol_type = other DNA
caagaacaactgggcagagc

SEQ ID NO: 98 - DNA, 19 bp, synthetic construct
  source 1..19: mol_type = other DNA
ggcctctgtccaccttcac

SEQ ID NO: 99 - DNA, 16 bp, synthetic construct
  source 1..16: mol_type = other DNA
ataggagccaggcgca

SEQ ID NO: 100 - DNA, 18 bp, synthetic construct
  source 1..18: mol_type = other DNA
ccagctccccatagtcca

SEQ ID NO: 101 - DNA, 20 bp, synthetic construct
  source 1..20: mol_type = other DNA
gagctgacgttctctcagca

SEQ ID NO: 102 - DNA, 19 bp, synthetic construct
  source 1..19: mol_type = other DNA
tacctccggggaacagcat

SEQ ID NO: 103 - DNA, 22 bp, synthetic construct
  source 1..22: mol_type = other DNA
caggcatgagtacctgtatggc

SEQ ID NO: 104 - DNA, 18 bp, synthetic construct
  source 1..18: mol_type = other DNA
catggtcaggtgaggggt

SEQ ID NO: 105 - DNA, 20 bp, synthetic construct
  source 1..20: mol_type = other DNA
tggttcacgctcctgtatga

SEQ ID NO: 106 - DNA, 18 bp, synthetic construct
  source 1..18: mol_type = other DNA
ctctgtttgctgcccagc

SEQ ID NO: 107 - DNA, 18 bp, synthetic construct
  source 1..18: mol_type = other DNA
acggtcactcgcatcctg

SEQ ID NO: 108 - DNA, 20 bp, synthetic construct
  source 1..20: mol_type = other DNA
ggtccagtcaggtctacgcc

SEQ ID NO: 109 - DNA, 20 bp, synthetic construct
  source 1..20: mol_type = other DNA
caattcatacgcgggatagc

SEQ ID NO: 110 - DNA, 18 bp, synthetic construct
  source 1..18: mol_type = other DNA
ccatgaaaaccgctgcat

SEQ ID NO: 111 - DNA, 17 bp, synthetic construct
  source 1..17: mol_type = other DNA
tcacacgccgaaacctg

SEQ ID NO: 112 - DNA, 21 bp, synthetic construct
  source 1..21: mol_type = other DNA
ggcccttcatgctcatcatag

SEQ ID NO: 113 - DNA, 18 bp, synthetic construct
  source 1..18: mol_type = other DNA
gcagcagtggattctcgg

SEQ ID NO: 114 - DNA, 24 bp, synthetic construct
  source 1..24: mol_type = other DNA
gcaggtaagggttaatctcaatca

SEQ ID NO: 115 - DNA, 18 bp, synthetic construct
  source 1..18: mol_type = other DNA
catggtagctggctggga

SEQ ID NO: 116 - DNA, 20 bp, synthetic construct
  source 1..20: mol_type = other DNA
aaggctgtcgagtcagcatt

SEQ ID NO: 117 - DNA, 23 bp, synthetic construct
  source 1..23: mol_type = other DNA
gggacctcttcaatgtcaatgat

SEQ ID NO: 118 - DNA, 23 bp, synthetic construct
  source 1..23: mol_type = other DNA
tccacacattgtttggtgtagtg

SEQ ID NO: 119 - DNA, 22 bp, synthetic construct
  source 1..22: mol_type = other DNA
ggtgcctgatgacatctacaaa

SEQ ID NO: 120 - DNA, 19 bp, synthetic construct
  source 1..19: mol_type = other DNA
gcagagtccacctgagagc

SEQ ID NO: 121 - DNA, 20 bp, synthetic construct
  source 1..20: mol_type = other DNA
gatcagggtcagcgagaagc

SEQ ID NO: 122 - DNA, 20 bp, synthetic construct
  source 1..20: mol_type = other DNA
ccgccagatcttgtagtgcc

SEQ ID NO: 123 - DNA, 20 bp, synthetic construct
  source 1..20: mol_type = other DNA
ttagcctgccctctttggac

SEQ ID NO: 124 - DNA, 20 bp, synthetic construct
  source 1..20: mol_type = other DNA
tcagaagggaggcagacagt

SEQ ID NO: 125 - DNA, 20 bp, synthetic construct
  source 1..20: mol_type = other DNA
agcaagcctcacatgtttcc

SEQ ID NO: 126 - DNA, 21 bp, synthetic construct
  source 1..21: mol_type = other DNA
caaggtaattacacgctctgc

SEQ ID NO: 127 - DNA, 20 bp, synthetic construct
  source 1..20: mol_type = other DNA
agctcatcaccaaggtcagc

SEQ ID NO: 128 - DNA, 20 bp, synthetic construct
  source 1..20: mol_type = other DNA
tcgtggtatacttgcccagc

SEQ ID NO: 129 - DNA, 21 bp, synthetic construct
  source 1..21: mol_type = other DNA
tactgccaagtcatccgtgtc

SEQ ID NO: 130 - DNA, 20 bp, synthetic construct
  source 1..20: mol_type = other DNA
tctccatcaggtgggccttc

SEQ ID NO: 131 - DNA, 22 bp, synthetic construct
  source 1..22: mol_type = other DNA
cctgatgctcatggagtgttcc

SEQ ID NO: 132 - DNA, 22 bp, synthetic construct
  source 1..22: mol_type = other DNA
ggaaagtggaggtgctgcacac

SEQ ID NO: 133 - DNA, 21 bp, synthetic construct
  source 1..21: mol_type = other DNA
ttctgctccctgtcagcttcc

SEQ ID NO: 134 - DNA, 21 bp, synthetic construct
  source 1..21: mol_type = other DNA
tcccagaaaccctttggtccg

SEQ ID NO: 135 - DNA, 22 bp, synthetic construct
  source 1..22: mol_type = other DNA
atgtcagtgggacttgtgctgc

SEQ ID NO: 136 - DNA, 22 bp, synthetic construct
  source 1..22: mol_type = other DNA
aactcagccaccatgaaccagg

SEQ ID NO: 137 - DNA, 19 bp, synthetic construct
  source 1..19: mol_type = other DNA
gaagtgcccttggttcgga

SEQ ID NO: 138 - DNA, 20 bp, synthetic construct
  source 1..20: mol_type = other DNA
gcagggtagcgattcgagtt

SEQ ID NO: 139 - DNA, 22 bp, synthetic construct
  source 1..22: mol_type = other DNA
actcaatggaagccacacatgg

SEQ ID NO: 140 - DNA, 22 bp, synthetic construct
  source 1..22: mol_type = other DNA
tacggctctgtctggtttgagg

SEQ ID NO: 141 - DNA, 20 bp, synthetic construct
  source 1..20: mol_type = other DNA
cgtggtcatcctctcctacg

SEQ ID NO: 142 - DNA, 19 bp, synthetic construct
  source 1..19: mol_type = other DNA
accttctggaagctctggc

SEQ ID NO: 143 - DNA, 23 bp, synthetic construct
  source 1..23: mol_type = other DNA
gttggcctttctgatcatattgc

SEQ ID NO: 144 - DNA, 20 bp, synthetic construct
  source 1..20: mol_type = other DNA
ctgcacagctggttcttccc

SEQ ID NO: 145 - DNA, 22 bp, synthetic construct
  source 1..22: mol_type = other DNA
atgttccttcagtcgtcaatgc

SEQ ID NO: 146 - DNA, 21 bp, synthetic construct
  source 1..21: mol_type = other DNA
ttgctgcactctgcaataact

SEQ ID NO: 147 - DNA, 22 bp, synthetic construct
  source 1..22: mol_type = other DNA
atctgtgccacatcagccacca

SEQ ID NO: 148 - DNA, 22 bp, synthetic construct
  source 1..22: mol_type = other DNA
aaggtggtgtccttctcagcca

SEQ ID NO: 149 - DNA, 22 bp, synthetic construct
  source 1..22: mol_type = other DNA
cagcacatcagagcagagaaag

SEQ ID NO: 150 - DNA, 20 bp, synthetic construct
  source 1..20: mol_type = other DNA
agcaaaaggctgagcttcaa

SEQ ID NO: 151 - DNA, 20 bp, synthetic construct
  source 1..20: mol_type = other DNA
cagggaggagaagcagaagc

SEQ ID NO: 152 - DNA, 20 bp, synthetic construct
  source 1..20: mol_type = other DNA
aggcggctcaatcttgaagt

SEQ ID NO: 153 - DNA, 22 bp, synthetic construct
  source 1..22: mol_type = other DNA
atgtcagacgaatgctagatgg

SEQ ID NO: 154 - DNA, 20 bp, synthetic construct
  source 1..20: mol_type = other DNA
ccaagttcttgaatcgtgcc

SEQ ID NO: 155 - DNA, 20 bp, synthetic construct
  source 1..20: mol_type = other DNA
gcgttttggaaagataccca

SEQ ID NO: 156 - DNA, 18 bp, synthetic construct
  source 1..18: mol_type = other DNA
ttgccaatctggacacca

SEQ ID NO: 157 - DNA, 23 bp, synthetic construct
  source 1..23: mol_type = other DNA
agctctattgcttggaacatggg

SEQ ID NO: 158 - DNA, 21 bp, synthetic construct
  source 1..21: mol_type = other DNA
cccctccaccaatggtcttgt

SEQ ID NO: 159 - DNA, 23 bp, synthetic construct
  source 1..23: mol_type = other DNA
gcgtgaaattgtccatattcaga

SEQ ID NO: 160 - DNA, 22 bp, synthetic construct
  source 1..22: mol_type = other DNA
aatcatctcccagaacttggct

SEQ ID NO: 161 - DNA, 24 bp, synthetic construct
  source 1..24: mol_type = other DNA
gcctgacaatttcatctttggtca

SEQ ID NO: 162 - DNA, 20 bp, synthetic construct
  source 1..20: mol_type = other DNA
caggcagtcgcagttttcac

SEQ ID NO: 163 - DNA, 18 bp, synthetic construct
  source 1..18: mol_type = other DNA
cagctggaccgcatctct

SEQ ID NO: 164 - DNA, 18 bp, synthetic construct
  source 1..18: mol_type = other DNA
agatccaccaggatggca

SEQ ID NO: 165 - DNA, 22 bp, synthetic construct
  source 1..22: mol_type = other DNA
atatgattgttgggaaggcatg

SEQ ID NO: 166 - DNA, 22 bp, synthetic construct
  source 1..22: mol_type = other DNA
aagtcctcttcttcgattccaa

SEQ ID NO: 167 - DNA, 20 bp, synthetic construct
  source 1..20: mol_type = other DNA
ttctgggacctggcactaag

SEQ ID NO: 168 - DNA, 21 bp, synthetic construct
  source 1..21: mol_type = other DNA
atcaccaaccactctggtgtc

SEQ ID NO: 169 - DNA, 22 bp, synthetic construct
  source 1..22: mol_type = other DNA
ggagaaaagatccatgaggaca

SEQ ID NO: 170 - DNA, 18 bp, synthetic construct
  source 1..18: mol_type = other DNA
cccagcaatggagtgaca

SEQ ID NO: 171 - DNA, 20 bp, synthetic construct
  source 1..20: mol_type = other DNA
cttgccttgctgctctacct

SEQ ID NO: 172 - DNA, 22 bp, synthetic construct
  source 1..22: mol_type = other DNA
tccatgaacttcaccacttcgt

SEQ ID NO: 173 - DNA, 19 bp, synthetic construct
  source 1..19: mol_type = other DNA
ctgacgatggcctggagtg

SEQ ID NO: 174 - DNA, 20 bp, synthetic construct
  source 1..20: mol_type = other DNA
cgggtaccggatcatgagga

(19) United States
(12) Patent Application Publication
(10) Pub. No.: US 2026/0317834 A1
(43) Pub. Date: Oct. 15, 2026

(54) METHOD FOR PERSONALIZED CANCER TREATMENT FOR A CANCER PATIENT

(71) Applicant: ONCOin Co., Ltd., Chuncheon-si (KR)

(72) Inventor: Yang Sik JEONG, Wonju-si (KR)

(21) Appl. No.: 19/682,063

(22) Filed: May 19, 2026

Related U.S. Application Data
(63) Continuation of application No. PCT/KR2024/017961, filed on Nov. 14, 2024.

(30) Foreign Application Priority Data
Nov. 20, 2023 (KR) 10-2023-0160411

(51) Int. Cl. C12Q 1/6886

(52) U.S. Cl. CPC C12Q 1/6886; C12Q 2600/106; C12Q 2600/136; C12Q 2600/158

(57) ABSTRACT

The present invention relates to a method for personalized cancer treatment for a cancer patient. More specifically, according to the present invention, expression changes of 87 target genes and mutations in some of these genes are quantified using a quantitative real-time PCR method, and the results are analyzed to conveniently profile expression patterns for individual cancer patients with high accuracy. Accordingly, the method for analyzing target gene expression patterns in cancer patients according to the present invention provides information that enables selective administration of existing anticancer agents or anticancer agents to be developed in the future to cancer patients, and thus can be utilized for patient-specific personalized treatment.

### CROSS-REFERENCE TO RELATED APPLICATIONS

This application is a bypass continuation application of PCT/KR2024/017961 filed 14 Nov. 2024, which claims the benefit of, and relies on the filing date of, Korean Patent Application No. 10-2023-0160411 filed 20 Nov. 2023, the entire disclosure of which is incorporated herein by reference.

### SEQUENCE LISTING

The instant application contains a Sequence Listing which has been submitted electronically in XML format and is hereby incorporated by reference in its entirety. Said XML copy, created on 12 Nov. 2024, is named PCT2024-014_Seq_list.xml and is 154,454 bytes in size.

### TECHNICAL FIELD

The present invention relates to a method for personalized cancer treatment for a cancer patient, and more specifically, to a method for selecting a personalized therapeutic agent for a cancer patient and a kit used therefor.

### BACKGROUND ART

Cancer is a disease that poses a significant threat to human health and life, accounting for approximately 13% of all deaths worldwide. In 2007, approximately 7.6 million people died from cancer globally. In the United States, approximately 1.4 million new cancer cases have been reported annually in recent years, and cancer is the second leading cause of death. According to statistics from the SEER report, the mortality rate for all cancer types in the United States increased from 195.4 per 100,000 in 1950 to 204.4 per 100,000 in 1978, and has steadily decreased to 184.0 per 100,000 in 2005. This decreasing trend appears to be attributable to early detection of cancer due to improved diagnostic technologies. Early detection and treatment play an important role in the prognosis and survival of patients across all cancer types.

Approximately 300 anticancer agents have been approved by the U.S. Food and Drug Administration (FDA) and the European Medicines Agency (EMA) and are currently used in clinical practice. These anticancer agents are approved for at least one type of cancer indication and are based on specific targets.

According to conventional cancer chemotherapy, anticancer agents are selected and administered based on the type and severity of cancer rather than on individual cancer patients. However, clinical outcomes generally show that the therapeutic effects of such chemotherapy vary significantly among patients, and various approaches have been proposed to overcome this limitation.

Accordingly, the present inventors have made efforts to develop a method capable of conveniently and accurately selecting a therapeutic agent suitable for an individual cancer patient. As a result, the inventors have found that expression changes of 87 genes, which are targets of anticancer agents approved by the U.S. FDA, and mutations in some of these genes can be quantified using a quantitative real-time PCR (qPCR) method, and the results can be analyzed to conveniently profile expression patterns for individual cancer patients with high accuracy, thereby enabling selection of a personalized therapeutic agent for a cancer patient, which has led to the present invention.

### PRIOR ART DOCUMENTS

### Patent Document

Korean Patent Registration No. 10-1371697

### DISCLOSURE OF THE INVENTION

### Technical Problem

An object of the present invention is to provide a kit for personalized selection of a therapeutic agent for a cancer patient using targets of anticancer agents, and a use thereof, as a method for more efficiently performing chemotherapy in cancer patients.

### Solution to Problem

In order to achieve the object of the present invention, the present invention provides a kit for personalized selection of a therapeutic agent for a cancer patient, comprising primers or probes that specifically hybridize to each of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB. The present invention also provides a method for personalized selection of a therapeutic agent for a cancer patient, comprising the steps of:
1) measuring expression of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB in a biological sample isolated from the cancer patient; and
2) selecting a therapeutic agent that acts on a target gene measured to be highly expressed in step 1).

In addition, the present invention provides a method for personalized screening of a therapeutic agent for a cancer patient, comprising the steps of:
1) isolating and proliferating cancer cells and normal cells from cancer tissue and normal tissue, respectively, isolated from the cancer patient;
2) treating the cancer cells of step 1) with a candidate anticancer agent;
3) isolating total RNA from the cancer cells and normal cells of step 1) and from the cancer cells treated with the candidate anticancer agent of step 2), and synthesizing cDNA using the total RNA as a template;
4) amplifying target genes using the cDNA synthesized in step 3) as a template and primers or probes that specifically hybridize to each of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB; and
5) measuring expression levels of the amplified target genes of step 4) and comparing the expression levels among the normal cells, the cancer cells, and the cancer cells treated with the candidate anticancer agent.

### Advantageous Effects of the Invention

According to the present invention, expression changes of 87 genes, which are targets of anticancer agents approved by the U.S. Food and Drug Administration (FDA), and mutations in some of these genes are quantified using a quantitative real-time PCR (qPCR) method, and the results are analyzed to conveniently profile expression patterns for individual cancer patients with high accuracy. Accordingly, the present invention can provide information for personalized selection of a therapeutic agent for a cancer patient. Therefore, the present invention can be utilized for patient-specific personalized treatment by selectively administering existing anticancer agents or anticancer agents to be developed in the future to cancer patients.

### BRIEF DESCRIPTION OF DRAWINGS

FIGS. 1a and 1b illustrate expression patterns of 87 target genes, as determined by quantitative real-time PCR (qPCR), for normal tissue and tumor tissue samples collected from three kidney cancer patients according to an embodiment of the present invention, wherein the first N and T denote normal tissue (N) and tumor tissue (T) samples from patient 1, the second N and T denote normal tissue (N) and tumor tissue (T) samples from patient 2, and the third N and T denote normal tissue (N) and tumor tissue (T) samples from patient 3.

FIGS. 2a and 2b illustrate expression patterns of 87 target genes, as determined by qPCR, for a normal human kidney cell line HK-2 and kidney cancer cell lines Caki-1, Caki-2, and ACHN, wherein the first bar graph represents the HK-2 cell line, the second bar graph represents the Caki-1 cell line, the third bar graph represents the Caki-2 cell line, and the fourth bar graph represents the ACHN cell line.

FIG. 3 is a graph showing cell viability after treatment of kidney cancer cell lines Caki-1, Caki-2, and ACHN with regorafenib at various concentrations.

FIG. 4 is a graph showing tumor size after administration of regorafenib in an ACHN xenograft mouse model.

FIG. 5 illustrates expression patterns of 87 target genes, as determined by qPCR, for normal tissue and ascites samples collected from a gastric cancer patient with malignant ascites according to an embodiment of the present invention, wherein the first bar graph represents the ascites sample and the second to sixth bar graphs represent the normal tissue samples.

FIG. 6 is a graph showing cell viability after treatment of cancer cells derived from an ascites sample of a gastric cancer patient with malignant ascites according to an embodiment of the present invention with carfilzomib, or with ramucirumab and paclitaxel, at various concentrations.

### BEST MODE FOR CARRYING OUT THE INVENTION

Hereinafter, the present invention will be described in more detail.

The present invention provides a kit for personalized selection of a therapeutic agent for a cancer patient, comprising primers or probes that specifically hybridize to each of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

In the present invention, the target genes may include at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB, or may include all of the target genes, but is not limited thereto.

In the present invention, the kit may be a kit for quantitative real-time PCR.

As used herein, the term “primer” refers to a short nucleic acid sequence having a free 3′ hydroxyl group, which is capable of forming base pairs with a complementary template and functions as a starting point for template strand replication. That is, a primer refers to a single-stranded oligonucleotide capable of initiating template-directed DNA synthesis under suitable conditions in an appropriate buffer (e.g., in the presence of four different nucleoside triphosphates and a polymerase such as DNA polymerase) and at an appropriate temperature.

The primers of the present invention may be chemically synthesized using a phosphoramidite solid support method or other methods well known in the art. In addition, the primers may be modified (e.g., by addition, deletion, or substitution) using various means known in the art, provided that such modifications do not adversely affect detection of the target genes. The primers need not be completely complementary to the template, but should be sufficiently complementary to hybridize to the template. Non-limiting examples of such modifications include methylation, capping, substitution with one or more analogs of natural nucleotides, and modifications of internucleotide linkages, such as uncharged linkages (e.g., methyl phosphonate, phosphotriester, phosphoramidate, carbamate, and the like) or charged linkages (e.g., phosphorothioate, phosphorodithioate, and the like). The nucleic acids may further comprise one or more additional covalently bound moieties, such as proteins (e.g., nucleases, toxins, antibodies, signal peptides, poly-L-lysine, and the like), intercalators (e.g., acridine, psoralen, and the like), chelators (e.g., metals, radioactive metals, iron, oxidative metals, and the like), and alkylating agents. The nucleic acid sequences of the present invention may also be modified with labels capable of directly or indirectly providing a detectable signal. Examples of such labels include radioactive isotopes, fluorescent molecules, biotin, and the like.

As used herein, the term “probe” refers to a linear oligomer of natural or modified monomers or linkages, including deoxyribonucleotides and ribonucleotides, which is capable of specifically hybridizing to a target nucleotide sequence, and may be naturally occurring or synthetically prepared.

The nucleotide sequences of the targets of the present invention, which are to be referenced for preparation of the primers or probes, may be obtained from GenBank, and primers or probes may be designed based on these sequences.

For example, as a set of primers that specifically hybridize to each of the target genes of the present invention, the primer sets shown in Table 1 below may be used, and specifically may be used for quantitative real-time PCR.

Specifically, primers that specifically hybridize to each of the target genes ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB may be selected from the group consisting of primers represented by SEQ ID NOs: 1 to 174 in Table 1 below.

Specifically, each primer that specifically hybridizes to each of the target genes ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB may be provided as primer sets, each comprising a pair of primers consisting of SEQ ID NOs: 1 and 2; SEQ ID NOs: 3 and 4; SEQ ID NOs: 5 and 6; SEQ ID NOs: 7 and 8; SEQ ID NOs: 9 and 10; SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; SEQ ID NOs: 29 and 30; SEQ ID NOs: 31 and 32; SEQ ID NOs: 33 and 34; SEQ ID NOs: 35 and 36; SEQ ID NOs: 37 and 38; SEQ ID NOs: 39 and 40; SEQ ID NOs: 41 and 42; SEQ ID NOs: 43 and 44; SEQ ID NOs: 45 and 46; SEQ ID NOs: 47 and 48; SEQ ID NOs: 49 and 50; SEQ ID NOs: 51 and 52; SEQ ID NOs: 53 and 54; SEQ ID NOs: 55 and 56; SEQ ID NOs: 57 and 58; SEQ ID NOs: 59 and 60; SEQ ID NOs: 61 and 62; SEQ ID NOs: 63 and 64; SEQ ID NOs: 65 and 66; SEQ ID NOs: 67 and 68; SEQ ID NOs: 69 and 70; SEQ ID NOs: 71 and 72; SEQ ID NOs: 73 and 74; SEQ ID NOs: 75 and 76; SEQ ID NOs: 77 and 78; SEQ ID NOs: 79 and 80; SEQ ID NOs: 81 and 82; SEQ ID NOs: 83 and 84; SEQ ID NOs: 85 and 86; SEQ ID NOs: 87 and 88; SEQ ID NOs: 89 and 90; SEQ ID NOs: 91 and 92; SEQ ID NOs: 93 and 94; SEQ ID NOs: 95 and 96; SEQ ID NOs: 97 and 98; SEQ ID NOs: 99 and 100; SEQ ID NOs: 101 and 102; SEQ ID NOs: 103 and 104; SEQ ID NOs: 105 and 106; SEQ ID NOs: 107 and 108; SEQ ID NOs: 109 and 110; SEQ ID NOs: 111 and 112; SEQ ID NOs: 113 and 114; SEQ ID NOs: 115 and 116; SEQ ID NOs: 117 and 118; SEQ ID NOs: 119 and 120; SEQ ID NOs: 121 and 122; SEQ ID NOs: 123 and 124; SEQ ID NOs: 125 and 126; SEQ ID NOs: 127 and 128; SEQ ID NOs: 129 and 130; SEQ ID NOs: 131 and 132; SEQ ID NOs: 133 and 134; SEQ ID NOs: 135 and 136; SEQ ID NOs: 137 and 138; SEQ ID NOs: 139 and 140; SEQ ID NOs: 141 and 142; SEQ ID NOs: 143 and 144; SEQ ID NOs: 145 and 146; SEQ ID NOs: 147 and 148; SEQ ID NOs: 149 and 150; SEQ ID NOs: 151 and 152; SEQ ID NOs: 153 and 154; SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 167 and 168; SEQ ID NOs: 169 and 170; SEQ ID NOs: 171 and 172; and SEQ ID NOs: 173 and 174.

In the present invention, the cancer may be kidney cancer, gastric cancer, lung cancer, breast cancer, ovarian cancer, liver cancer, bronchial cancer, lymphoma, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer, ureter cancer, or hematological cancer, and may specifically be kidney cancer, but is not limited thereto.

In addition, the cancer patient may, regardless of cancer type, exhibit symptoms of advanced-stage cancer, such as ascites or pleural effusion (BAL fluid), but is not limited thereto.

In the present invention, the kit may comprise DNA polymerase, dNTPs, a buffer, and the like for performing a PCR amplification reaction. The kit may further comprise a user manual describing optimal reaction conditions. The manual is a printed material describing how to use the kit, for example, preparation of reverse transcription buffer and PCR buffer, and the reaction conditions provided. The manual may be provided in the form of a booklet or leaflet, a label attached to the kit, or instructions printed on a surface of a package containing the kit. The manual may also include information disclosed or provided through an electronic medium, such as the Internet.

In addition, the present invention provides a method for personalized selection of a therapeutic agent for a cancer patient, comprising the steps of:
1) measuring expression of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB in a biological sample isolated from the cancer patient; and
2) selecting a therapeutic agent that acts on a target gene measured to be highly expressed in step 1).

In the method of the present invention, in step 1), the biological sample may include various types of biological samples, and specifically may be blood, serum, plasma, tissue, cells, lymph, bone marrow fluid, saliva, urine, feces, ocular fluid, semen, brain extract, cerebrospinal fluid, synovial fluid, thymic fluid, ascites, or amniotic fluid, more specifically tissue or cells, and even more specifically cancer tissue or cancer cells.

In the method of the present invention, in step 1), the target genes may include at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB, or may include all of the target genes, but is not limited thereto.

In the method of the present invention, in step 1), expression of the gene may be measured by quantitative real-time PCR.

In the method of the present invention, in step 2), the therapeutic agent that acts on the target gene may be an anticancer agent approved by the U.S. Food and Drug Administration (FDA) or the European Medicines Agency (EMA) and used in clinical practice, but is not limited thereto. For example, anticancer agents approved by the U.S. FDA and used in clinical practice are described in The Author(s), BMC Systems Biology 2017, 11(Suppl 5):87.

In addition, the present invention provides a method for personalized screening of a therapeutic agent for a cancer patient, comprising the steps of:
1) isolating and proliferating cancer cells and normal cells from cancer tissue and normal tissue, respectively, isolated from the cancer patient;
2) treating the cancer cells of step 1) with a candidate anticancer agent;
3) isolating total RNA from the cancer cells and normal cells of step 1) and from the cancer cells treated with the candidate anticancer agent of step 2), and synthesizing cDNA using the total RNA as a template;
4) amplifying target genes using the cDNA synthesized in step 3) as a template and primers or probes that specifically hybridize to each of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB; and
5) measuring expression levels of the amplified target genes of step 4) and comparing the expression levels among the normal cells, the cancer cells, and the cancer cells treated with the candidate anticancer agent.

In the method of the present invention, in step 1), the method for isolating cancer cells and normal cells from cancer tissue and normal tissue isolated from the cancer patient may be any method known in the art. For example, cancer tissue or normal tissue may be dissociated using a tissue-dissociating enzyme or a mechanical method, and the dissociated cells may be separated according to cell size, density, or surface characteristics using density gradient centrifugation, cell sorting, magnetic separation, or the like.

In the method of the present invention, in step 2), the candidate anticancer agent may include any substance, molecule, element, compound, entity, or a combination thereof. For example, although not limited thereto, the candidate anticancer agent may include proteins, polypeptides, small organic molecules, polysaccharides, polynucleotides, and the like. The candidate anticancer agent may also be a natural product, a synthetic compound, a chemical compound, or a combination of two or more substances. Specific examples include, but are not limited to, polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purines, pyrimidines, derivatives thereof, structural analogs thereof, or combinations thereof. The candidate anticancer agent may be a synthetic substance, and other candidate agents may be natural substances.

In the method of the present invention, in step 3), total RNA may be isolated using any method known in the art, for example, a phenol extraction method.

In the method of the present invention, in step 4), the target genes may include at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB, or may include all of the target genes, but is not limited thereto.

In the method of the present invention, in step 4), the primers or probes may be primers or probes for quantitative real-time PCR.

In the method of the present invention, in step 5), a candidate anticancer agent may be selected as a personalized therapeutic agent for a cancer patient when expression levels of the target genes are increased in cancer cells compared to normal cells and decreased in cancer cells treated with the candidate anticancer agent compared to untreated cancer cells.

Hereinafter, the present invention will be described in detail with reference to the following Examples.

However, the following Examples are provided for illustrative purposes only, and the scope of the present invention is not limited thereto.

### <Example 1> Preparation of Kidney Cancer Patient Samples

Normal tissue and tumor tissue samples obtained from three kidney cancer patients were acquired from the specimen bank of the Department of Pathology at Wonju Severance Christian Hospital.

Three pairs of normal tissue and tumor tissue samples were placed in cryo-tubes and stored at −70° C. until RNA extraction. Total RNA was extracted using a commercially available kit, RNeasy Midi Kit (Qiagen, Chatsworth, CA, USA), according to the manufacturer's protocol. The quantity and quality of the extracted total RNA were evaluated using ultraviolet spectrophotometry (DU 530, Beckman, USA).

Reverse transcription was performed using 2 μg of total RNA in a reaction volume of 20 μL under conditions of 2 units of DNase I (4.2 μM MgCl₂). The reaction mixture was composed of 2 μg of total RNA, 3.68 μL of 50 mM MgCl₂, 0.96 μL of DNase I, and DEPC-treated water to a final volume of 20 μL.

The DNase I-treated sample was subjected to reverse transcription using Superscript II Reverse Transcription Kit (Invitrogen, Cat. No. 18064-071) under the following conditions: 20 μL of 5× First Strand Buffer, 10 μL of 100 mM DTT, 20 μL of 10 mM dNTPs, 5 μL of pdN6 (1.6 μg/μL), 0.5 μL of RTase (200 U/μL), 20 μL of RNA, and 24.5 μL of DEPC-treated water, to a total reaction volume of 100 μL.

The reverse transcription reaction was performed under the following temperature conditions: 25° C. for 10 min 42° C. for 50 min 72° C. for 10 min, and a hold at 4° C.

After completion of the reverse transcription reaction, the cDNA was diluted with DEPC-treated water to a concentration of 5 ng/μL.

### <Example 2> Quantitative Real-Time PCR Analysis for Personalized Selection of a Therapeutic Agent for a Cancer Patient

For personalized selection of a therapeutic agent for a cancer patient, quantitative real-time PCR (qPCR) analysis was performed using the cDNA obtained in <Example 1> as a template and primers for 87 target genes shown in Table 1 below, thereby establishing mRNA expression profiles of the 87 targets.

Specifically, 87 molecular biological targets suitable for the present invention were selected from therapeutic targets of approximately 300 anticancer agents approved by the U.S. Food and Drug Administration (FDA) or the European Medicines Agency (EMA), including kidney cancer therapeutics such as the mTOR inhibitor everolimus, and qPCR primers for these targets were designed as shown in Table 1 below.

TABLE 1

| | | | GenBank | |
|---|---|---|---|---|
| | Gene | Public | accession | |
| Number | name | availability | number | Primer sequence(5′→3′) |
| 1 | ACPP | No | NM_001099 | (Forward) |
| | | | | TTGTACTTTGAGAAGGGGGAGT |
| | | | | (SEQ ID NO: 1) |
| | | | | (Reverse) |
| | | | | GGTAGCATGAGGGGATACGG |
| | | | | (SEQ ID NO: 2) |
| 2 | ADA | No | NM_000022 | (Forward) |
| | | | | AAGAGGAGTTTAAAAGGCTGAACA |
| | | | | (SEQ ID NO: 3) |
| | | | | (Reverse) AAGGTGGCATCCCATAGGC |
| | | | | (SEQ ID NO: 4) |
| 3 | ALK | No | NM_004304 | (Forward) |
| | | | | ATTGGAGACTTCGGGATGGC |
| | | | | (SEQ ID NO: 5) |
| | | | | (Reverse) |
| | | | | CTTAACTGGCAGCATGGCAC |
| | | | | (SEQ ID NO: 6) |
| 4 | BCR- | No | NM_005157 | (Forward) |
| | ABL-1 | | | GGCCAGTGGAGATAACACTCTAA |
| | | | | (SEQ ID NO: 7) |
| | | | | (Reverse) |
| | | | | CACCATTCCCCATTGTGATTATA |
| | | | | (SEQ ID NO: 8) |
| 5 | BTK | No | NM_000061 | (Forward) |
| | | | | AGAAGCACCTTTTCAGCACCA |
| | | | | (SEQ ID NO: 9) |
| | | | | (Reverse) |
| | | | | ACACTGGATATTTGAGCCTGGAT |
| | | | | (SEQ ID NO: 10) |
| 6 | CD19 | No | NM_001178098 | (Forward) |
| | | | | ATGAAGAGCTGACCCAGCCG |
| | | | | (SEQ ID NO: 11) |
| | | | | (Reverse) |
| | | | | CCTCATAGGACTGGGACCCT |
| | | | | (SEQ ID NO: 12) |
| 7 | CD3D | No | NM_000732 | (Forward) |
| | | | | GAACATAGCACGTTTCTCTCTGG |
| | | | | (SEQ ID NO: 13) |
| | | | | (Reverse) |
| | | | | GTATCTTGAAGGGGCTCACTTG |
| | | | | (SEQ ID NO: 14) |
| 8 | CD52 | No | NM_001803 | (Forward) |
| | | | | TTCCTCTTCCTCCTACTCACCA |
| | | | | (SEQ ID NO: 15) |
| | | | | (Reverse) |
| | | | | CTGGTGTCGTTTTGTCCTGA |
| | | | | (SEQ ID NO: 16) |
| 9 | CDK4 | No | NM_000075 | (Forward) |
| | | | | TTTGTGGCCCTCAAGAGTGT |
| | | | | (SEQ ID NO: 17) |
| | | | | (Reverse) |
| | | | | ACGTCCATCAGCCGGACAAC |
| | | | | (SEQ ID NO: 18) |
| 10 | CDK6 | No | NM_001145306 | (Forward) |
| | | | | TCTTGCTCCAGTCCAGCTAC |
| | | | | (SEQ ID NO: 19) |
| | | | | (Reverse) |
| | | | | GGGAGTCCAATCACGTCCAAG |
| | | | | (SEQ ID NO: 20) |
| 11 | C-MET | No | NM_001127500 | (Forward) |
| | | | | GGACATCAGAGGGTCGCTT |
| | | | | (SEQ ID NO: 21) |
| | | | | (Reverse) |
| | | | | GATGGGAGTCCAGGAGAAAAT |
| | | | | (SEQ ID NO: 22) |
| 12 | CRBN | No | NM_016302 | (Forward) GACGCTGCGCACAACAT |
| | | | | (SEQ ID NO: 23) |
| | | | | (Reverse) |
| | | | | CTGGTCTTCAACTTCCATTTCATC |
| | | | | (SEQ ID NO: 24) |
| 13 | CTLA4 | No | NM_005214 | (Forward) |
| | | | | GTACCCACCGCCATACTACC |
| | | | | (SEQ ID NO: 25) |
| | | | | (Reverse) |
| | | | | GCATTTTCACATAGACCCCTG |
| | | | | (SEQ ID NO: 26) |
| 14 | CYP17A1 | No | NM_000102 | (Forward) |
| | | | | GTTCAAGGATGGCGATCAG |
| | | | | (SEQ ID NO: 27) |
| | | | | (Reverse) |
| | | | | GGCCAGCATATCACACAATGT |
| | | | | (SEQ ID NO: 28) |
| 15 | DDR2 | No | NM_001014796 | (Forward) |
| | | | | CCCAGGAAACTCCTAACTTTCAA |
| | | | | (SEQ ID NO: 29) |
| | | | | (Reverse) |
| | | | | TCCCTCCACTTCACAGAGATG |
| | | | | (SEQ ID NO: 30) |
| 16 | ERBB4 | No | NM_005235 | (Forward) |
| | | | | GAACAGCAGTACCGAGCCTT |
| | | | | (SEQ ID NO: 31) |
| | | | | (Reverse) |
| | | | | ACTTCTCGAACAGACCGCAG |
| | | | | (SEQ ID NO: 32) |
| 17 | FCGRIA | No | NM_00137880 | (Forward) TCCTTAAGCGCAGCCCT |
| | | | | (SEQ ID NO: 33) |
| | | | | (Reverse) |
| | | | | AGGACATGAAACCAGACAGGA |
| | | | | (SEQ ID NO: 34) |
| 18 | FGF1 | No | NM_000800 | (Forward) |
| | | | | GGGCTTTTATACGGCTCACAGAC |
| | | | | (SEQ ID NO: 35) |
| | | | | (Reverse) |
| | | | | AATGGTTCTCCTCCAGCCTT |
| | | | | (SEQ ID NO: 36) |
| 19 | FGFR1 | No | NM_023110 | (Forward) |
| | | | | GACGCAGGGGAGTATACGTG |
| | | | | (SEQ ID NO: 37) |
| | | | | (Reverse) |
| | | | | TCTCTTCCAGGGCTTCCAGA |
| | | | | (SEQ ID NO: 38) |
| 20 | FGFR2 | No | NM_000141 | (Forward) |
| | | | | CACGACCAAGAAGCCAGACT |
| | | | | (SEQ ID NO: 39) |
| | | | | (Reverse) |
| | | | | TGGACTCAGCCGAAACTGTTA |
| | | | | (SEQ ID NO: 40) |
| 21 | FGFR3 | No | NM_000142 | (Forward) |
| | | | | GAATGCCTCCCACGAGGAC |
| | | | | (SEQ ID NO: 41) |
| | | | | (Reverse) |
| | | | | GAGGATGGAGCGTCTGTCAC |
| | | | | (SEQ ID NO: 42) |
| 22 | FRK | No | NM_002031 | (Forward) |
| | | | | CCGATGTATGGTCATTTGGAAT |
| | | | | (SEQ ID NO: 43) |
| | | | | (Reverse) CTGGATTACCTGGGCACCT |
| | | | | (SEQ ID NO: 44) |
| 23 | GNRH1 | No | NM_000825 | (Forward) |
| | | | | GGTTGGTCAACTGGCAGAAAC |
| | | | | (SEQ ID NO: 45) |
| | | | | (Reverse) |
| | | | | AGAGCTCCTTTCAGGTCTCG |
| | | | | (SEQ ID NO: 46) |
| 24 | GNRHR | No | NM_000406 | (Forward) |
| | | | | ATCTTCACCCTGACACGGG |
| | | | | (SEQ ID NO: 47) |
| | | | | (Reverse) |
| | | | | TTAGAGTCTTCAGCCGTGCTC |
| | | | | (SEQ ID NO: 48) |
| 25 | HDAC2 | No | NM_001527 | (Forward) |
| | | | | TCTGCTACTACTACGACGGTGAT |
| | | | | (SEQ ID NO: 49) |
| | | | | (Reverse) |
| | | | | CAGTGGCTTTATGGGGCCTA |
| | | | | (SEQ ID NO: 50) |
| 26 | HDAC3 | No | NM_001355039 | (Forward) |
| | | | | TGGCATTGATGACCAGAGTTACA |
| | | | | (SEQ ID NO: 51) |
| | | | | (Reverse) |
| | | | | TGCACGTGGGTTGGTAGAAG |
| | | | | (SEQ ID NO: 52) |
| 27 | HPRT1 | No | NM_000194 | (Forward) |
| | | | | CTGGCGTCGTGATTAGTGAT |
| | | | | (SEQ ID NO: 53) |
| | | | | (Reverse) |
| | | | | GAGCAAGACGTTCAGTCCTGT |
| | | | | (SEQ ID NO: 54) |
| 28 | IFNAR1 | No | NM_001384498 | (Forward) |
| | | | | TGCTGCGAAAGTCTTCTTGAG |
| | | | | (SEQ ID NO: 55) |
| | | | | (Reverse) |
| | | | | TGGCTGTTCAGAGAAATACTCATC |
| | | | | (SEQ ID NO: 56) |
| 29 | IFNAR2 | No | NM_207585 | (Forward) |
| | | | | TGAGCCAGAATGCCTTCATC |
| | | | | (SEQ ID NO: 57) |
| | | | | (Reverse) |
| | | | | AGATTCATCTGTGTAATCAGGCG |
| | | | | (SEQ ID NO: 58) |
| 30 | IL2RA | No | NM_000417 | (Forward) |
| | | | | GAATGCAAGAGAGGTTTCCGC |
| | | | | (SEQ ID NO: 59) |
| | | | | (Reverse) |
| | | | | TGTTCCGAGTGGCAGAGCTT |
| | | | | (SEQ ID NO: 60) |
| 31 | IL2RB | No | NM_000878 | (Forward) |
| | | | | ACACTTCCTGCCAAGTCCA |
| | | | | (SEQ ID NO: 61) |
| | | | | (Reverse) ATGCTTGACTCACGGGGA |
| | | | | (SEQ ID NO: 62) |
| 32 | IL2RG | No | NM_000206 | (Forward) |
| | | | | ATTTCTGGCTGGAACGGACG |
| | | | | (SEQ ID NO: 63) |
| | | | | (Reverse) |
| | | | | GCCAGTCCCTTAGACACACC |
| | | | | (SEQ ID NO: 64) |
| 33 | ITK | No | NM_005546 | (Forward) |
| | | | | CCTCCTACTCCTGAAGACAACAG |
| | | | | (SEQ ID NO: 65) |
| | | | | (Reverse) |
| | | | | TGAGGATCATTGGTTTGGTAGTC |
| | | | | (SEQ ID NO: 66) |
| 34 | JAK1 | No | NM_002227 | (Forward) |
| | | | | CACAGGCATGCCGTATCTCT |
| | | | | (SEQ ID NO: 67) |
| | | | | (Reverse) |
| | | | | CCAGAGCTTGGTGTTCTCGT |
| | | | | (SEQ ID NO: 68) |
| 35 | JAK2 | No | NM_004972 | (Forward) |
| | | | | TGCTGCTTCTAAAGCTTGTGGTAT |
| | | | | (SEQ ID NO: 69) |
| | | | | (Reverse) |
| | | | | GGAAGACATGGTTGGGTGGAT |
| | | | | (SEQ ID NO: 70) |
| 36 | LDLR | No | NM_000527 | (Forward) |
| | | | | TGACAATGTCTCACCAAGCTCT |
| | | | | (SEQ ID NO: 71) |
| | | | | (Reverse) |
| | | | | GAGGACAATGGACAGAGCCC |
| | | | | (SEQ ID NO: 72) |
| 37 | LHCGR | No | NM_000233 | (Forward) |
| | | | | CCATCTCAAGCTTTCAGAGGA |
| | | | | (SEQ ID NO: 73) |
| | | | | (Reverse) |
| | | | | TAGCTTCTATCCTTTCCAGGGA |
| | | | | (SEQ ID NO: 74) |
| 38 | LIMK1 | No | NM_002314 | (Forward) |
| | | | | TGACACACCGTGAGACAGGT |
| | | | | (SEQ ID NO: 75) |
| | | | | (Reverse) |
| | | | | GCATGACCTTCACCTCCTTGA |
| | | | | (SEQ ID NO: 76) |
| 39 | MAPIA | No | NM_002373 | (Forward) |
| | | | | GGGAGAATCTTCAGGTGACTC |
| | | | | (SEQ ID NO: 77) |
| | | | | (Reverse) |
| | | | | CCAGGACCAGGACATTCAGT |
| | | | | (SEQ ID NO: 78) |
| 40 | MAP2 | No | NM_002374 | (Forward) |
| | | | | CCATCACCTGCCTCAGAAC |
| | | | | (SEQ ID NO: 79) |
| | | | | (Reverse) |
| | | | | AATACACTGGGAGCCAGAGC |
| | | | | (SEQ ID NO: 80) |
| 41 | MAP2K1 | No | NM_002755 | (Forward) |
| | | | | AGCTCTGCGGAGACCAACT |
| | | | | (SEQ ID NO: 81) |
| | | | | (Reverse) |
| | | | | GCTGCTGCTCATCAAGCTCT |
| | | | | (SEQ ID NO: 82) |
| 42 | MAP2K2 | No | NM_030662 | (Forward) |
| | | | | TATATTGTGAACGAGCCACCTCC |
| | | | | (SEQ ID NO: 83) |
| | | | | (Reverse) |
| | | | | TGGTTTGTGAGCATCTTCAGGT |
| | | | | (SEQ ID NO: 84) |
| 43 | MAPK11 | No | NM_002751 | (Forward) |
| | | | | CGAGGACTTCAGCGAAGTGTA |
| | | | | (SEQ ID NO: 85) |
| | | | | (Reverse) |
| | | | | GCCGAGTGGATGTACTTCAG |
| | | | | (SEQ ID NO: 86) |
| 44 | NEK11 | No | NM_024800 | (Forward) |
| | | | | CCAAACGAGGAGAGGAATTAAAG |
| | | | | (SEQ ID NO: 87) |
| | | | | (Reverse) |
| | | | | GCCTGTACAGTTTCATTTGGATTT |
| | | | | (SEQ ID NO: 88) |
| 45 | NR3C1 | No | NM_000176 | (Forward) |
| | | | | AAGTTTTCTTCAAAAGAGCAGTGG |
| | | | | (SEQ ID NO: 89) |
| | | | | (Reverse) |
| | | | | TCGATGATGCAATCATTCCT |
| | | | | (SEQ ID NO: 90) |
| 46 | NTRK1 | No | NM_001012331 | (Forward) TCTGGAGCTCCGTGATCTG |
| | | | | (SEQ ID NO: 91) |
| | | | | (Reverse) |
| | | | | AGTGAAATGGAAGGCATCTGG |
| | | | | (SEQ ID NO: 92) |
| 47 | PARP1 | No | NM_001618 | (Forward) |
| | | | | ACTTTGCTGGGATCCTGTCC |
| | | | | (SEQ ID NO: 93) |
| | | | | (Reverse) |
| | | | | CAAACATGTAGCCTGTCACGG |
| | | | | (SEQ ID NO: 94) |
| 48 | PARP2 | No | NM_005484 | (Forward) |
| | | | | TCACAGGTTACATGTTTGGGA |
| | | | | (SEQ ID NO: 95) |
| | | | | (Reverse) |
| | | | | TTCATTACACTGACCTAGAGCTACC |
| | | | | (SEQ ID NO: 96) |
| 49 | PARP3 | No | NM_001003931 | (Forward) |
| | | | | CAAGAACAACTGGGCAGAGC |
| | | | | (SEQ ID NO: 97) |
| | | | | (Reverse) GGCCTCTGTCCACCTTCAC |
| | | | | (SEQ ID NO: 98) |
| 50 | PDCD1 | No | NM_005018 | (Forward) ATAGGAGCCAGGCGCA |
| | | | | (SEQ ID NO: 99) |
| | | | | (Reverse) CCAGCTCCCCATAGTCCA |
| | | | | (SEQ ID NO: 100) |
| 51 | PGF | No | NM_002632 | (Forward) |
| | | | | GAGCTGACGTTCTCTCAGCA |
| | | | | (SEQ ID NO: 101) |
| | | | | (Reverse) TACCTCCGGGGAACAGCAT |
| | | | | (SEQ ID NO: 102) |
| 52 | PIK3CD | No | NM_005026 | (Forward) |
| | | | | CAGGCATGAGTACCTGTATGGC |
| | | | | (SEQ ID NO: 103) |
| | | | | (Reverse) CATGGTCAGGTGAGGGGT |
| | | | | (SEQ ID NO: 104) |
| 53 | PRLR | No | NM_000949 | (Forward) |
| | | | | TGGTTCACGCTCCTGTATGA |
| | | | | (SEQ ID NO: 105) |
| | | | | (Reverse) CTCTGTTTGCTGCCCAGC |
| | | | | (SEQ ID NO: 106) |
| 54 | PSMB10 | No | NM_002801 | (Forward) ACGGTCACTCGCATCCTG |
| | | | | (SEQ ID NO: 107) |
| | | | | (Reverse) |
| | | | | GGTCCAGTCAGGTCTACGCC |
| | | | | (SEQ ID NO: 108) |
| 55 | PSMB1 | No | NM_002793 | (Forward) |
| | | | | CAATTCATACGCGGGATAGC |
| | | | | (SEQ ID NO: 109) |
| | | | | (Reverse) CCATGAAAACCGCTGCAT |
| | | | | (SEQ ID NO: 110) |
| 56 | PSMB2 | No | NM_002794 | (Forward) TCACACGCCGAAACCTG |
| | | | | (SEQ ID NO: 111) |
| | | | | (Reverse) |
| | | | | GGCCCTTCATGCTCATCATAG |
| | | | | (SEQ ID NO: 112) |
| 57 | PSMB8 | No | NM_004159 | (Forward) GCAGCAGTGGATTCTCGG |
| | | | | (SEQ ID NO: 113) |
| | | | | (Reverse) |
| | | | | GCAGGTAAGGGTTAATCTCAATCA |
| | | | | (SEQ ID NO: 114) |
| 58 | PSMB9 | No | NM_002800 | (Forward) CATGGTAGCTGGCTGGGA |
| | | | | (SEQ ID NO: 115) |
| | | | | (Reverse) |
| | | | | AAGGCTGTCGAGTCAGCATT |
| | | | | (SEQ ID NO: 116) |
| 59 | PSMD1 | No | NM_002807 | (Forward) |
| | | | | GGGACCTCTTCAATGTCAATGAT |
| | | | | (SEQ ID NO: 117) |
| | | | | (Reverse) |
| | | | | TCCACACATTGTTTGGTGTAGTG |
| | | | | (SEQ ID NO: 118) |
| 60 | PSMD2 | No | NM_002808 | (Forward) |
| | | | | GGTGCCTGATGACATCTACAAA |
| | | | | (SEQ ID NO: 119) |
| | | | | (Reverse) |
| | | | | GCAGAGTCCACCTGAGAGC |
| | | | | (SEQ ID NO: 120) |
| 61 | PTK6 | No | NM_005975 | (Forward) |
| | | | | GATCAGGGTCAGCGAGAAGC |
| | | | | (SEQ ID NO: 121) |
| | | | | (Reverse) |
| | | | | CCGCCAGATCTTGTAGTGCC |
| | | | | (SEQ ID NO: 122) |
| 62 | RARA | No | NM_000964 | (Forward) |
| | | | | TTAGCCTGCCCTCTTTGGAC |
| | | | | (SEQ ID NO: 123) |
| | | | | (Reverse) |
| | | | | TCAGAAGGGAGGCAGACAGT |
| | | | | (SEQ ID NO: 124) |
| 63 | RARB | No | NM_000965 | (Forward) |
| | | | | AGCAAGCCTCACATGTTTCC |
| | | | | (SEQ ID NO: 125) |
| | | | | (Reverse) |
| | | | | CAAGGTAATTACACGCTCTGC |
| | | | | (SEQ ID NO: 126) |
| 64 | RARG | No | NM_000966 | (Forward) |
| | | | | AGCTCATCACCAAGGTCAGC |
| | | | | (SEQ ID NO: 127) |
| | | | | (Reverse) |
| | | | | TCGTGGTATACTTGCCCAGC |
| | | | | (SEQ ID NO: 128) |
| 65 | RPL3 | No | NM_000967 | (Forward) |
| | | | | TACTGCCAAGTCATCCGTGTC |
| | | | | (SEQ ID NO: 129) |
| | | | | (Reverse) |
| | | | | TCTCCATCAGGTGGGCCTTC |
| | | | | (SEQ ID NO: 130) |
| 66 | SH2B3 | No | NM_005475 | (Forward) |
| | | | | CCTGATGCTCATGGAGTGTTCC |
| | | | | (SEQ ID NO: 131) |
| | | | | (Reverse) |
| | | | | GGAAAGTGGAGGTGCTGCACAC |
| | | | | (SEQ ID NO: 132) |
| 67 | SIK1 | No | NM_173354 | (Forward) |
| | | | | TTCTGCTCCCTGTCAGCTTCC |
| | | | | (SEQ ID NO: 133) |
| | | | | (Reverse) |
| | | | | TCCCAGAAACCCTTTGGTCCG |
| | | | | (SEQ ID NO: 134) |
| 68 | SLC2A2 | No | NM_000340 | (Forward) |
| | | | | ATGTCAGTGGGACTTGTGCTGC |
| | | | | (SEQ ID NO: 135) |
| | | | | (Reverse) |
| | | | | AACTCAGCCACCATGAACCAGG |
| | | | | (SEQ ID NO: 136) |
| 69 | SMO | No | NM_005631 | (Forward) |
| | | | | GAAGTGCCCTTGGTTCGGA |
| | | | | (SEQ ID NO: 137) |
| | | | | (Reverse) |
| | | | | GCAGGGTAGCGATTCGAGTT |
| | | | | (SEQ ID NO: 138) |
| 70 | SSTR2 | No | NM_001050 | (Forward) |
| | | | | ACTCAATGGAAGCCACACATGG |
| | | | | (SEQ ID NO: 139) |
| | | | | (Reverse) |
| | | | | TACGGCTCTGTCTGGTTTGAGG |
| | | | | (SEQ ID NO: 140) |
| 71 | SSTR5 | No | NM_001053 | (Forward) |
| | | | | CGTGGTCATCCTCTCCTACG |
| | | | | (SEQ ID NO: 141) |
| | | | | (Reverse) ACCTTCTGGAAGCTCTGGC |
| | | | | (SEQ ID NO: 142) |
| 72 | TEK | No | NM_000459 | (Forward) |
| | | | | GTTGGCCTTTCTGATCATATTGC |
| | | | | (SEQ ID NO: 143) |
| | | | | (Reverse) |
| | | | | CTGCACAGCTGGTTCTTCCC |
| | | | | (SEQ ID NO: 144) |
| 73 | TLR8 | No | NM_016610 | (Forward) |
| | | | | ATGTTCCTTCAGTCGTCAATGC |
| | | | | (SEQ ID NO: 145) |
| | | | | (Reverse) |
| | | | | TTGCTGCACTCTGCAATAACT |
| | | | | (SEQ ID NO: 146) |
| 74 | TNFSF8 | No | NM_001244 | (Forward) |
| | | | | ATCTGTGCCACATCAGCCACCA |
| | | | | (SEQ ID NO: 147) |
| | | | | (Reverse) |
| | | | | AAGGTGGTGTCCTTCTCAGCCA |
| | | | | (SEQ ID NO: 148) |
| 75 | TNFSF11 | No | NM_003701 | (Forward) |
| | | | | CAGCACATCAGAGCAGAGAAAG |
| | | | | (SEQ ID NO: 149) |
| | | | | (Reverse) |
| | | | | AGCAAAAGGCTGAGCTTCAA |
| | | | | (SEQ ID NO: 150) |
| 76 | TOP1MT | No | NM_052963 | (Forward) |
| | | | | CAGGGAGGAGAAGCAGAAGC |
| | | | | (SEQ ID NO: 151) |
| | | | | (Reverse) |
| | | | | AGGCGGCTCAATCTTGAAGT |
| | | | | (SEQ ID NO: 152) |
| 77 | TOP2B | No | NM_001330700 | (Forward) |
| | | | | ATGTCAGACGAATGCTAGATGG |
| | | | | (SEQ ID NO: 153) |
| | | | | (Reverse) |
| | | | | CCAAGTTCTTGAATCGTGCC |
| | | | | (SEQ ID NO: 154) |
| 78 | TUBAIA | No | NM_006009 | (Forward) |
| | | | | GCGTTTTGGAAAGATACCCA |
| | | | | (SEQ ID NO: 155) |
| | | | | (Reverse) TTGCCAATCTGGACACCA |
| | | | | (SEQ ID NO: 156) |
| 79 | TUBA4A | No | NM_006000 | (Forward) |
| | | | | AGCTCTATTGCTTGGAACATGGG |
| | | | | (SEQ ID NO: 157) |
| | | | | (Reverse) |
| | | | | CCCCTCCACCAATGGTCTTGT |
| | | | | (SEQ ID NO: 158) |
| 80 | TUBB1 | No | NM_030773 | (Forward) |
| | | | | GCGTGAAATTGTCCATATTCAGA |
| | | | | (SEQ ID NO: 159) |
| | | | | (Reverse) |
| | | | | AATCATCTCCCAGAACTTGGCT |
| | | | | (SEQ ID NO: 160) |
| 81 | TUBB3 | No | NM_006086 | (Forward) |
| | | | | GCCTGACAATTTCATCTTTGGTCA |
| | | | | (SEQ ID NO: 161) |
| | | | | (Reverse) |
| | | | | CAGGCAGTCGCAGTTTTCAC |
| | | | | (SEQ ID NO: 162) |
| 82 | TUBB | No | NM_001293212 | (Forward) CAGCTGGACCGCATCTCT |
| | | | | (SEQ ID NO: 163) |
| | | | | (Reverse) AGATCCACCAGGATGGCA |
| | | | | (SEQ ID NO: 164) |
| 83 | TUBD1 | No | NM_016261 | (Forward) |
| | | | | ATATGATTGTTGGGAAGGCATG |
| | | | | (SEQ ID NO: 165) |
| | | | | (Reverse) |
| | | | | AAGTCCTCTTCTTCGATTCCAA |
| | | | | (SEQ ID NO: 166) |
| 84 | TUBE1 | No | NM_016262 | (Forward) |
| | | | | TTCTGGGACCTGGCACTAAG |
| | | | | (SEQ ID NO: 167) |
| | | | | (Reverse) |
| | | | | ATCACCAACCACTCTGGTGTC |
| | | | | (SEQ ID NO: 168) |
| 85 | TUBG1 | No | NM_001070 | (Forward) |
| | | | | GGAGAAAAGATCCATGAGGACA |
| | | | | (SEQ ID NO: 169) |
| | | | | (Reverse) CCCAGCAATGGAGTGACA |
| | | | | (SEQ ID NO: 170) |
| 86 | VEGFA | No | NM_001025366 | (Forward) |
| | | | | CTTGCCTTGCTGCTCTACCT |
| | | | | (SEQ ID NO: 171) |
| | | | | (Reverse) |
| | | | | TCCATGAACTTCACCACTTCGT |
| | | | | (SEQ ID NO: 172) |
| 87 | VEGFB | No | NM_001243733 | (Forward) |
| | | | | CTGACGATGGCCTGGAGTG |
| | | | | (SEQ ID NO: 173) |
| | | | | (Reverse) |
| | | | | CGGGTACCGGATCATGAGGA |
| | | | | (SEQ ID NO: 174) |

The qPCR reaction mixture was prepared using the primers described above according to the composition shown in Table 2 below. 10 μL of the qPCR reaction mixture was dispensed into a 384-well plate, and qPCR was performed using an ABI 7900 HT instrument under the conditions shown in Table 3 below. The amount of each gene was quantified using SDS 2.4 software.

TABLE 2

| reaction solution | 1X (μl) |
|---|---|
| 2X SYBR | 5 |
| 0.25 μM Primer Mix | 1.2 |
| RNase-free water | 2.8 |
| Template | 1 |

TABLE 3
PCR condition

| Stage | Step | Time | | Temp |
|---|---|---|---|---|
| Hold stage | 1 | 2 min | | 50° C. |
| | 2 (Initial heating) | 10 min | | 95° C. |
| PCR stage | 1 (Denaturation) | 15 sec | 40 cycles | 95° C. |
| | 2 (Annealing) | 1 min | | 60° C. |

| | | | |
|---|---|---|---|
| Melt curve stage | 1 | 15 sec | 95° C. |
| | 2 | 1 min | 60° C. |
| | 3 (Dissociation) | 15 sec | 95° C. |

### <Experimental Example 1> Expression Profiles of Target Genes in Kidney Cancer Patients by qPCR Analysis

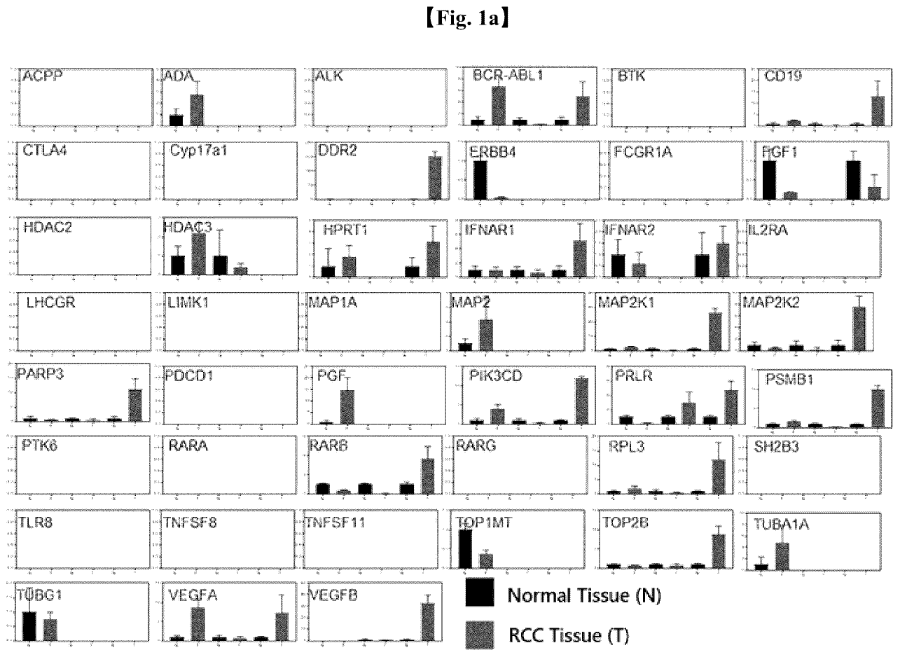

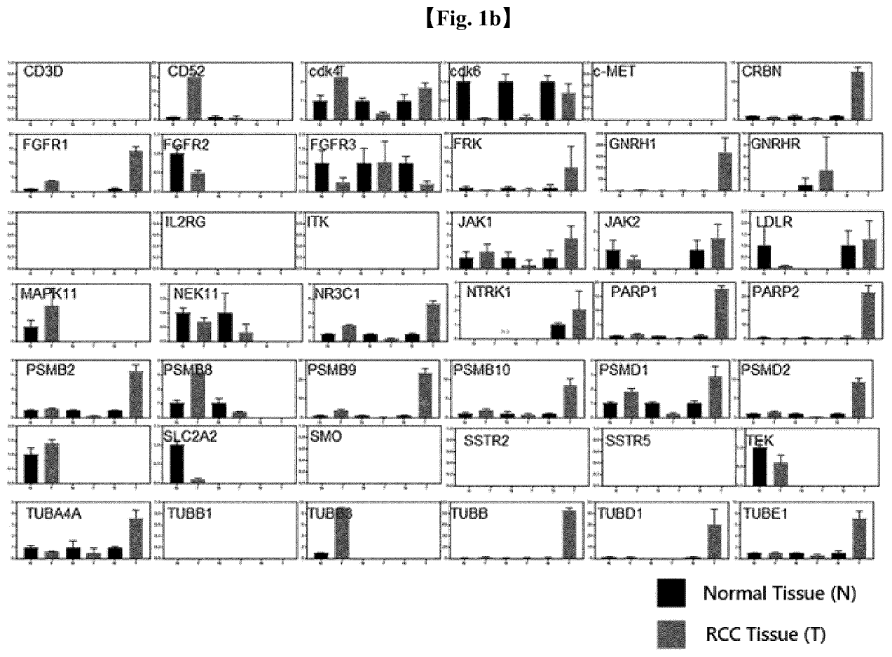

Using normal tissue and tumor tissue samples obtained from three kidney cancer patients in <Example 1>, qPCR was performed according to the method described in <Example 2> to establish expression profiles of 87 target genes (FIGS. 1a and 1b).

As a result, as shown in FIGS. 1a and 1b, differences in expression patterns of the 87 target genes were observed between normal tissues and tumor tissues in the kidney cancer patients.

These results indicate that, even in the same type of kidney cancer, expression levels of target genes for anticancer agents may vary among patients, and suggest that effective treatment may be achieved by identifying patient-specific expression profiles.

### <Experimental Example 2> Expression Profiles of Target Genes in Kidney Cancer Cell Lines by qPCR Analysis and Selection of Personalized Anticancer Agents

<2-1> Expression Profiles of Target Genes in Kidney Cancer Cell Lines by qPCR Analysis

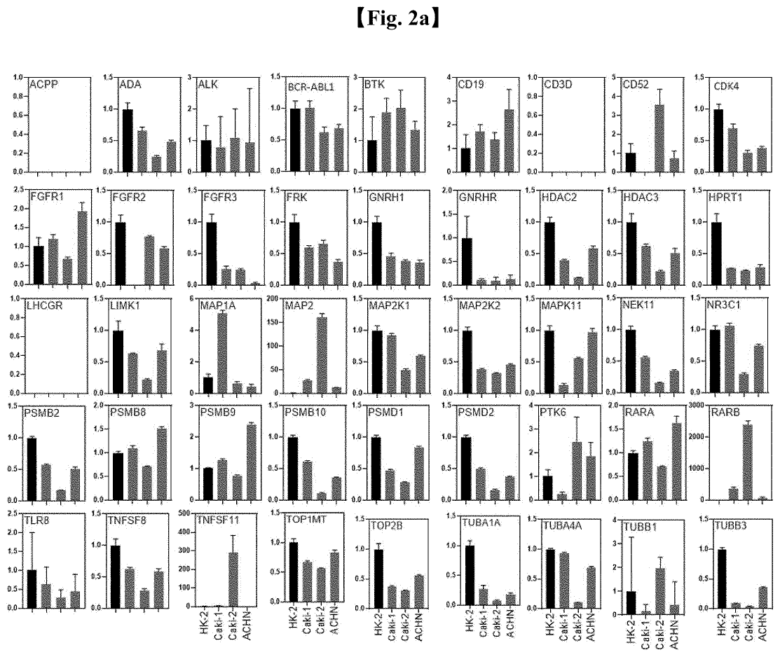

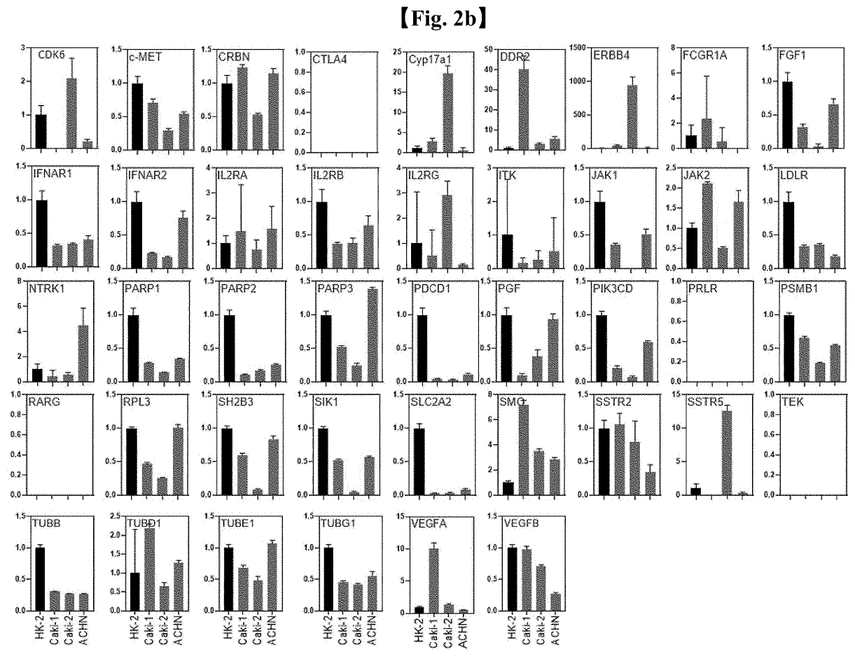

Samples were prepared using three kidney cancer cell lines, Caki-1, Caki-2, and ACHN, in the same manner as described in <Example 1>, and qPCR was performed according to the method described in <Example 2> to establish expression profiles of 87 target genes (FIGS. 2a and 2b). As a control, HK-2 cells, a normal human kidney-derived cell line, were used.

As a result, as shown in FIGS. 2a and 2b, differences in expression patterns of the 87 target genes were observed among the three kidney cancer cell lines. In particular, expression of DDR2 was markedly high in the Caki-1 cell line, whereas expression was not high in the Caki-2 and ACHN cell lines.

### <2-2> In Vitro and In Vivo Validation of Personalized Anticancer Agent Selection

To select personalized anticancer agents based on gene expression patterns, anticancer agents were administered to the three kidney cancer cell lines of <Example 2-1>, and sensitivity to the anticancer agents was evaluated.

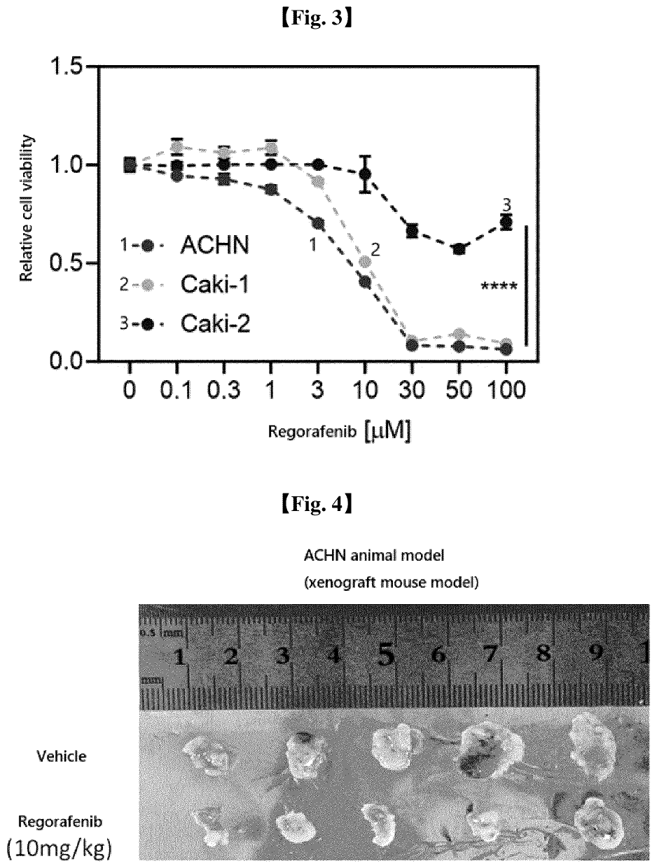

Specifically, Caki-1, Caki-2, and ACHN kidney cancer cell lines were seeded in 96-well plates at a density of 4,000 to 5,000 cells per well and cultured for 24 hours, followed by treatment with regorafenib, an FDA-approved therapeutic agent for metastatic kidney cancer, at concentrations ranging from 0 to 100 μM. After 72 hours, the number of viable cells was quantified using sulforhodamine B (SRB) staining (FIG. 3).

As a result, as shown in FIG. 3, the Caki-1 cell line, which exhibited high DDR2 expression, showed lower sensitivity to regorafenib, whereas the Caki-2 and ACHN cell lines, which exhibited low DDR2 expression, showed higher sensitivity to regorafenib.

In addition, sensitivity to regorafenib was evaluated in an ACHN xenograft mouse model.

Specifically, animal experiments were performed in accordance with the National Institutes of Health Guide for the Care and Use of Laboratory Animals and were approved by the Institutional Animal Care and Use Committee (IACUC) of Wonju Severance Christian Hospital (approval No. YWC-190917-3). For establishment of the tumor model, 5×10⁶ ACHN cells were prepared in a syringe and subcutaneously injected into the right shoulder of 5-week-old male BALB/c nude mice (Orient Bio, Inc.) weighing 20-25 g. When the tumors reached a diameter of approximately 0.5 cm after 2 weeks, the control group (vehicle, n=5) received vehicle, and the treatment group (n=5) received 10 mg/kg regorafenib intravenously three times per week for a total of 4 weeks, followed by measurement of tumor size (FIG. 4).

As a result, as shown in FIG. 4, the treatment group exhibited a superior anticancer effect of regorafenib.

These results suggest that regorafenib may be a suitable anticancer agent for kidney cancer exhibiting low DDR2 expression.

### <Experimental Example 3> Expression Profiles of Target Genes in a Gastric Cancer Patient by qPCR Analysis and Selection of Personalized Anticancer Agents

<3-1> Expression Profiles of Target Genes in a Gastric Cancer Patient by qPCR Analysis

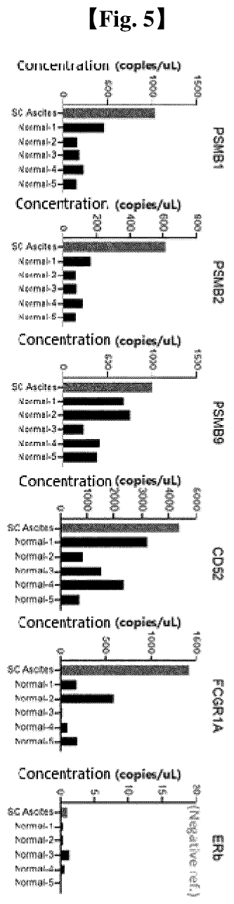

Ascites samples and normal tissue samples obtained from a gastric cancer patient exhibiting malignant ascites were acquired from the specimen bank of the Department of Pathology at Wonju Severance Christian Hospital. The samples were prepared in the same manner as described in <Example 1>, and qPCR was performed according to the method described in <Example 2> to profile the five target genes showing the highest expression levels among the 87 target genes (FIG. 5).

As a result, as shown in FIG. 5, high expression levels were observed for five target genes, namely PSMB1, PSMB2, PSMB9, CD52, and FCGR1A. Among these, PSMB1, PSMB2, and PSMB9 were identified as targets of carfilzomib.

### <3-2> In Vitro Validation of Personalized Anticancer Agent Selection

Anticancer agents were administered to gastric cancer cells derived from ascites samples of the gastric cancer patient exhibiting malignant ascites in <Example 3-1>, and sensitivity to the anticancer agents was evaluated.

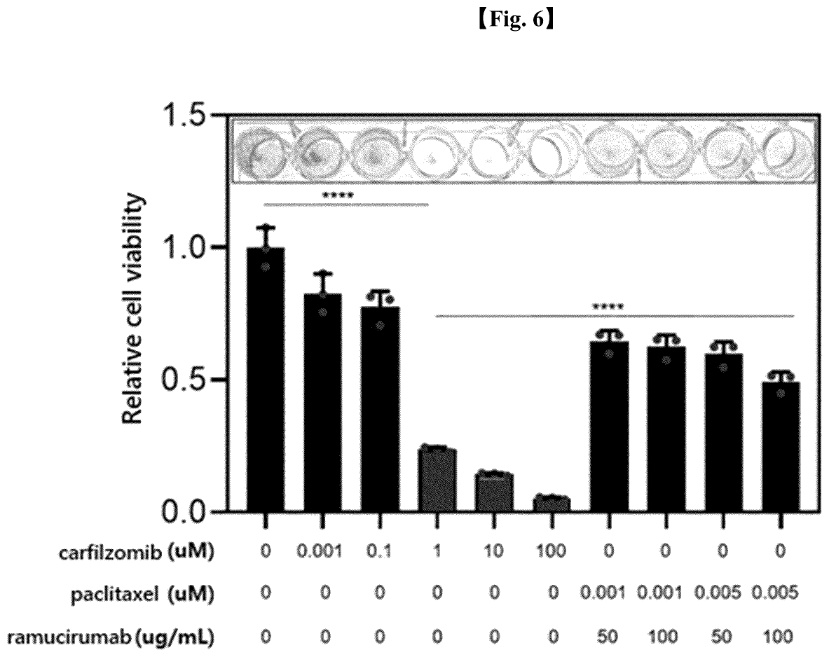

Specifically, gastric cancer cells derived from ascites samples of the patient were seeded at a density of 4,000 to 5,000 cells per well and cultured for 24 hours, followed by treatment with carfilzomib, which was identified as a therapeutic agent targeting overexpressed genes in <Example 3-1>, at concentrations ranging from 0 to 100 μM for 16 hours. As comparative groups, ramucirumab and paclitaxel, which had been administered to the patient, were each treated at concentrations ranging from 0 to 100 μM for 24 hours. After treatment, the number of viable cells was quantified using sulforhodamine B (SRB) staining (FIG. 6).

As a result, as shown in FIG. 6, the carfilzomib-treated group exhibited a significantly greater reduction in cell viability compared to the ramucirumab- and paclitaxel-treated groups.

These results suggest that, even in the same type of cancer, expression levels of the 87 target genes for anticancer agents may vary among patients, and that by quantifying and analyzing changes in expression and mutations of some of these genes using qPCR, personalized anticancer agents can be conveniently selected for individual cancer patients with high accuracy.

### INDUSTRIAL APPLICABILITY

The method for analyzing expression patterns of target genes in cancer patients according to the present invention provides information for selectively administering existing anticancer agents or anticancer agents to be developed in the future to individual cancer patients, and thus can be utilized for personalized treatment of patients.

1. A kit for selecting a personalized therapeutic agent for a cancer patient, comprising primers or probes that specifically hybridize to at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

2. The kit of claim 1, wherein the kit comprises primers or probes that specifically hybridize to at least ten target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

3. The kit of claim 2, wherein the kit comprises primers or probes that specifically hybridize to at least twenty target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

4. The kit of claim 3, wherein the kit comprises primers or probes that specifically hybridize to at least twenty-five target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

5. The kit of claim 4, wherein the kit comprises primers or probes that specifically hybridize to at least thirty-five target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

6. The kit of claim 5, wherein the kit comprises primers or probes that specifically hybridize to at least forty-five target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

7. The kit of claim 1, wherein the primers that specifically hybridize to each of the target genes are selected from the group consisting of primers represented by SEQ ID NOs: 1 to 174.

8. The kit of claim 1, wherein the kit is a kit for quantitative real-time PCR.

9. The kit of claim 1, wherein the cancer is selected from the group consisting of kidney cancer, gastric cancer, lung cancer, breast cancer, ovarian cancer, liver cancer, bronchial cancer, lymphoma, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer, ureter cancer, and hematological cancer.

10. A method for personalized selection of a therapeutic agent for a cancer patient, comprising the steps of:
1) measuring expression of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB in a biological sample isolated from the cancer patient; and
2) selecting a therapeutic agent that acts on the target gene measured to be highly expressed in step 1).

11. The method of claim 10, wherein the measuring expression of the gene is performed by quantitative real-time PCR.

12. A method for personalized screening of a therapeutic agent for a cancer patient, comprising the steps of:
1) isolating and proliferating cancer cells and normal cells from cancer tissue and normal tissue, respectively, isolated from the cancer patient;
2) treating the cancer cells of step 1) with a candidate anticancer agent;
3) isolating total RNA from the cancer cells and normal cells of step 1) and from the cancer cells treated with the candidate anticancer agent of step 2), and synthesizing cDNA using the total RNA as a template;
4) amplifying target genes using the cDNA synthesized in step 3) as a template and primers or probes that specifically hybridize to each of at least two target genes selected from the group consisting of ACPP, ADA, ALK, BCR-ABL-1, BTK, CDK4, CDK6, CD19, CD3D, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB; and
5) measuring expression levels of the amplified target genes of step 4) and comparing the expression levels of the target genes among the normal cells, the cancer cells, and the cancer cells treated with the candidate anticancer agent.

13. The method of claim 12, wherein the primers or probes are primers or probes for quantitative real-time PCR.

14. The method of claim 12, wherein a candidate agent is selected that shows an increase in expression levels of the target genes in cancer cells compared to normal cells and a decrease in expression levels of the target genes in cancer cells treated with the candidate anticancer agent compared to untreated cancer cells.
